(19) United States
(12) Patent Application Publication
(10) Pub. No.: US 2016/0058683 A1
(43) Pub. Date: Mar. 3, 2016

(54) Hair-Straightening Compositions and Methods

(71) Applicant: Cosphatech LLC, Miami, FL (US)

(72) Inventors: Sergio Adrian Engrassi, Buenos Aires (AR); Marc Adrien Pfeiffer, Miami, FL (US)

(21) Appl. No.: 14/840,827

(22) Filed: Aug. 31, 2015

Related U.S. Application Data
(60) Provisional application No. 62/044,263, filed on Aug. 31, 2014.

(51) Int. Cl. A61K 8/36; A61K 8/49; A45D 7/06; A61K 8/42; A61Q 5/02; A61K 8/365; A61Q 5/04

(52) U.S. Cl. CPC A61K 8/36; A61K 8/365; A61K 8/4973; A61Q 5/04; A61K 8/4913; A61K 8/42; A61Q 5/02; A45D 7/06; A61K 2800/30; A45D 2007/008

(57) ABSTRACT

A hair-straightening complex consisting essentially of (i) at least one glyoxyloyl derivative formed by reacting glyoxylic acid with at least one alpha, beta or gamma amino acid and/or an amino acid analogue, where the amino acid, or analogue thereof, is not derived from an animal source, (ii) at least one organic acid, (iii) optionally, at least one humectant, and (iv) at least one monoalkyl or dialkyl ether of sorbitol or 1,4-Dianhydrosorbitol. A method of straightening hair comprising the steps of applying the above hair-straightening complex to the hair and applying a force generally perpendicular to the length of the hair using a flat surface heated at a temperature of from about 380° F. to about 450° F.

### BACKGROUND OF THE INVENTION

Numerous treatments for modifying the shape and texture of human hair are known in the personal care arts, including application of keratin and keratin amino acids as well as the combination of heat and a composition comprising formalin. Around 2003, hair-straightening/smoothing with formaldehyde was popularized in Brazil. This approach begins with application of a solution containing formaldehyde to wavy and curly hair; the treated hair is blow-dried and flat ironed, producing smooth, shiny, and straight hair. Hair straighteners are sometimes also referred to in the art as hair relaxers.

In the United States, the Occupational Safety and Health Administration (OSHA), as well as state and local agencies and professional organizations, such as the American Conference of Governmental Industrial Hygienists (ACGIH), establish occupational exposure limits (OELs). Section 1910.1048 of Title 29 of the Code of Federal Regulations sets out the standards for airborne exposure to formaldehyde: the permissible exposure level (PEL) for an 8-hour time weighted average (TWA) is 0.75 parts per million (ppm); the action level (AL) for an 8-hour TWA is 0.5 ppm; the short-term exposure limit (STEL) is 2 ppm for a 15-minute TWA. If a product is capable of releasing formaldehyde at levels exceeding 0.5 ppm, the product label is required to contain the words “Potential Cancer Hazard.” ACGIH lists formaldehyde as a sensitizer and has set a ceiling limit of 0.3 ppm.

Federal agencies as well as State OSHA programs have responded to concerns relating to in-salon formaldehyde exposure from hair smoothing products that have been labeled as “formaldehyde-free.” State OSHA programs in Oregon and California as well as the Connecticut Department of Public Health have issued warnings about these products to salon owners, workers, and salon patrons.

The National Institute for Occupational Safety and Health (NIOSH) completed a Health Hazard Evaluation for Brazilian Blowout Acai Professional Smoothing Solution-Formaldehyde Free Smoothing Formula. Background (general area (GA) air samples, as well as personal breathing zone (PBZ) air samples were collected prior to and during treatment with the Brazilian Blowout smoothing product. More particularly, NIOSH Method 2016 was used to collect samples that were further analyzed using Environmental Protection Agency Method TO-11. Based on measured formaldehyde air concentrations and determination that the Brazilian Blowout smoothing product contained formaldehyde at greater than 0.1%, NIOSH concluded that OSHA's formaldehyde standard required the employer to alert workers about potential risks of formaldehyde-related health effects including cancer, irritation, and sensitization of the skin and respiratory system.

The Food and Drug Administration (FDA) issued a warning letter to the importer and distributor of Brazilian Blowout Acai Professional Smoothing Solution, finding the product to be “adulterated” and “misbranded” because it contains methylene glycol, which can release formaldehyde during the normal conditions of use, and because the label makes misleading statements (“Formaldehyde Free or “No Formaldehyde”).

The hair-straightening compositions and methods of the present invention meet the need for a haircare product that is does not emit formaldehyde above safe exposure levels and that does not contain animal-derived keratin.

### SUMMARY OF THE INVENTION

The present invention is directed to methods of straightening human hair comprising the step of applying to the hair a composition comprising a hair-straightening complex consisting essentially of
(i) at least one, preferably more a mixture of several, glyoxyloyl derivative(s) that is/are formed by reacting glyoxylic acid with at least one alpha, beta or gamma amino acid and/or an amino acid analogue, where the amino acid, or analogue thereof, is not derived from an animal source,
(ii) a first organic acid, preferably glyoxylic acid,
(iii) optionally, a second organic acid (in addition to glyoxylic acid), preferably a hydroxy acid or a keto acid having six or fewer carbon atoms,
(iv) optionally, at least one humectant, preferably a natural moisturizing factor, and
(v) at least one monoalkyl or dialkyl ether of sorbitol and/or 1,4-Dianhydrosorbitol
   wherein when a haircare product containing the above complex is used in a hair-straightening procedure that includes a step of applying heat to the hair, the user's/stylist's exposure to formaldehyde does not exceed 0.5 ppm on an 8-hour time-weighted average (TWA) basis.

### DESCRIPTION OF THE FIGURES

FIG. 1a shows hair tresses before application of the hair-straightening complexes of the present invention.

From left to right, FIG. 1b shows the hair-straightening efficacy of hair-straightening complexes of the present invention described in Examples 2, 3, and 1.

FIG. 2 shows the hair-straightening efficacy of a cationic emulsion base containing the hair-straightening complexes of Examples 2 (left) and 3 (right).

FIG. 3 shows the hair-straightening efficacy of a slightly anionic gel containing the hair-straightening complexes in Examples 2 (left) and 3 (right).

FIG. 4 shows the continued hair-straightening properties imparted by a cationic emulsion containing the hair-straightening complex of Example 3 after five washes.

FIG. 5 shows the continued hair-straightening properties imparted by a cationic emulsion containing the hair-straightening complex of Example 3 after 15, 25, 35 and 45 washes.

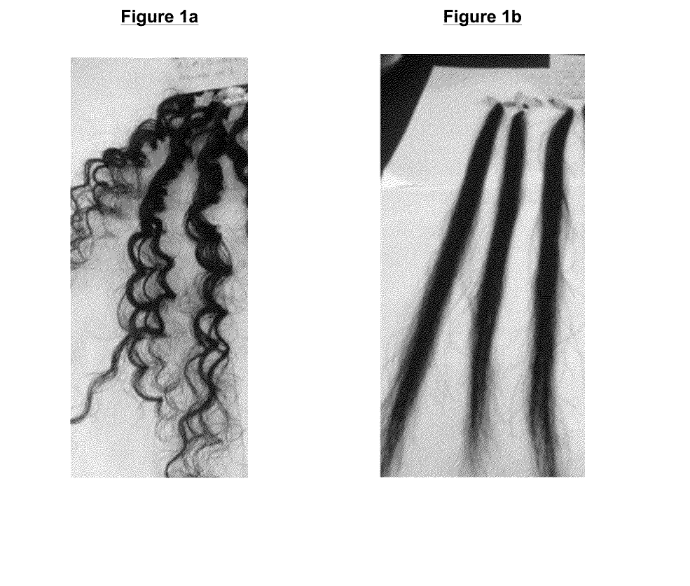

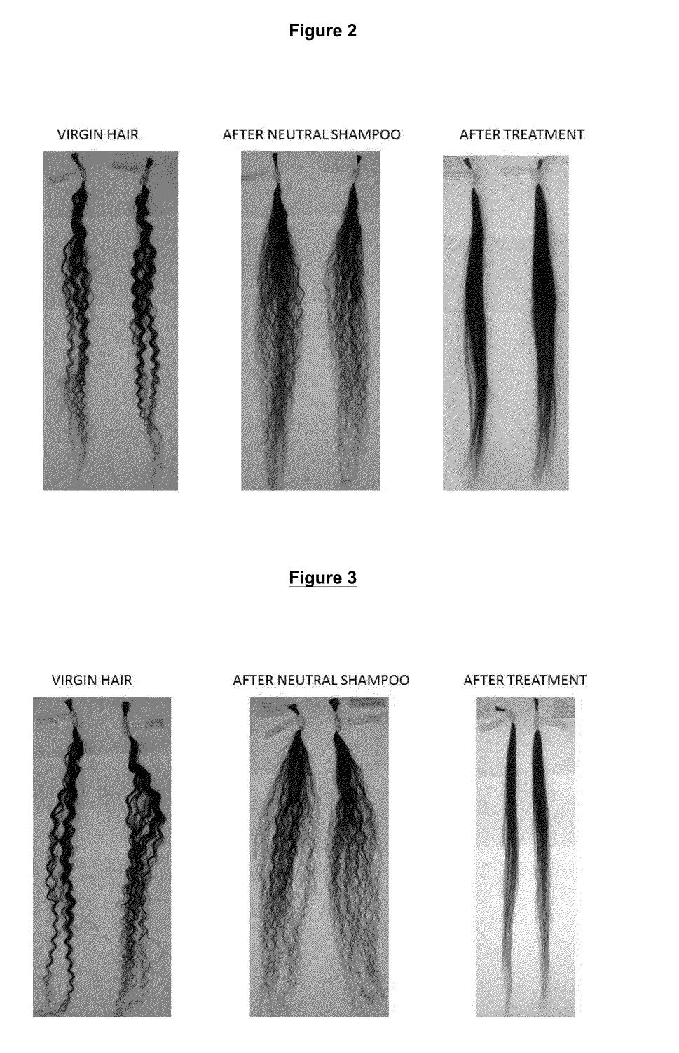

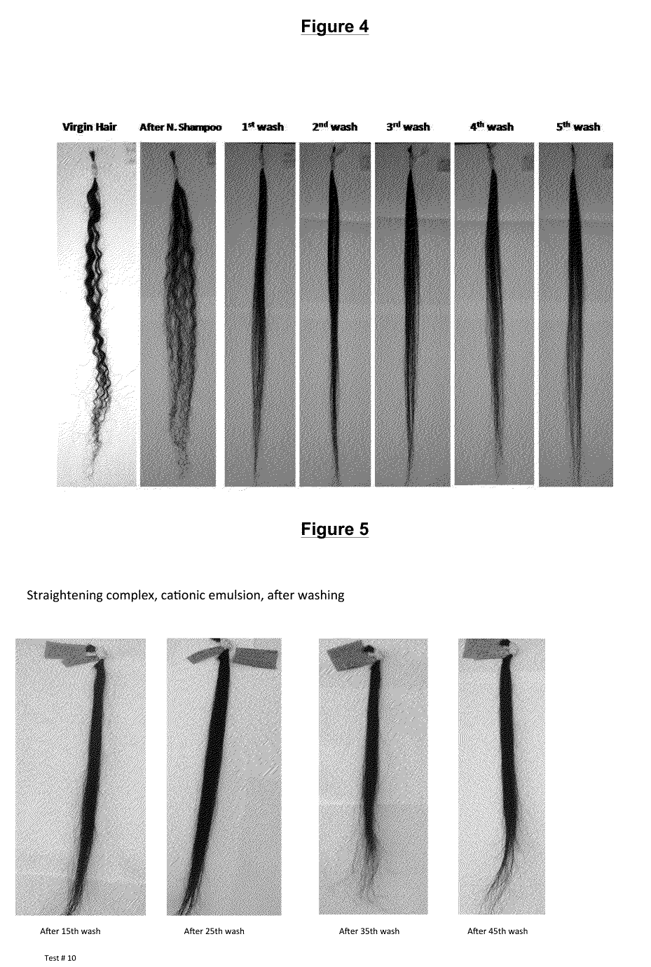

### DETAILED DESCRIPTION OF THE INVENTION

The inventive hair-straightening complex of the present invention is incorporated into a finished haircare product—a hair-straightening product—which can be in the form of a solution, a gel, a cream, a lotion, a paste or a mousse, and can be a single-phase (e.g., thickened aqueous dispersion or anhydrous gel) or multi-phase system (e.g., emulsion), where the phases are oil, water, and silicone.

As used herein with respect to the ingredients in hair-straightening products of the present invention, the term “consisting essentially of” means that in addition to the components of the inventive hair-straightening complex, the hair-straightening product may contain other ingredients suitable for application to human hair provided that such additional ingredient(s) does/do not generate formaldehyde at a level that exceeds 0.5 ppm on an 8-hour TWA basis.

Unless otherwise indicated, all percentages recited in connection with the composition of the hair-straightening composition are by weight with respect to the total weight of the composition.

As used herein in connection with a measured quantity, the term “about” refers to that variation in the measured quantity as would be expected by the person of ordinary skill in the art performing the measurement and exercising a level of care commensurate with the purpose of the measurement and the precision of the measuring equipment used, taking into account factors including experimental and rounding errors.

The hair-straightening complex of the present invention, and hair-straightening products containing the inventive hair-straightening complex, includes as a required component the reaction product of glyoxylic acid with at least one alpha, beta or gamma amino acid, or an analogue thereof, where the amino acid (or analogue thereof) is preferably selected from the group of (a) essential amino acids, (b) non-essential amino acids, (c) conditional amino acids and (d) acetyl amino acid derivatives.

The reaction product of glyoxylic acid with at least one alpha, beta or gamma amino acid, or an analogue thereof, is referred to herein as a glyoxyloyl derivative. The term glyoxyloyl derivative is to be understood to include glyoxyloyl esters and glyoxyloyl thioesters, and salts thereof.

An organic acid is an organic chemical compound containing one or more carboxyl radicals.

Glyoxylic acid is an organic acid having the empirical formula of C₂H₂O₃ and conforming to the structure:

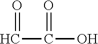

In some preferred embodiments of the present invention, glyoxylic acid is reacted with at least one essential amino acid (or analogue thereof) that is selected from the group consisting of Histidine, Isoleucine, Leucine, Lysine, Methionine, Phenylalanine, Threonine, Tryptophan, and Valine.

In other preferred embodiments of the present invention, glyoxylic acid is reacted with at least one non-essential amino acid (or analogue thereof) that is selected from the group consisting of Alanine, Asparagine, Aspartic Acid, and Glutamic Acid.

In still other preferred embodiments of the present invention, glyoxylic acid is reacted with at least one conditional amino acid (or analogue thereof) selected from the group consisting of Arginine, Cysteine, Glutamine, Tyrosine, Glycine, Ornithine, Proline, and Serine.

Selenocysteine is a cysteine analogue with a selenium-containing selenol group in place of the sulfur-containing thiol group and is yet another preferred amino acid that can be reacted with glyoxylic acid to produce a glyoxyloyl derivative useful in the compositions and methods of the present invention.

A preferred beta amino acid that can be reacted with glyoxylic acid to produce a glyoxyloyl derivative useful in the compositions and methods of the present invention is Beta-Alanine.

In certain embodiments, preferred glyoxyloyl derivatives useful in the compositions and methods of the present invention are produced by reacting an n-acetyl amino acid with glyoxylic acid. Preferred glyoxyloyl derivatives within the scope of this embodiment of the present invention include the reaction product of glyoxylic acid with an n-acetyl amino acid selected from the group consisting of N-Acetyl Methionine, N-Acetyl Cysteine, N-Acetyl Aspartic Acid, N-Acetyl Ornithine, N-Acetyl Lysine, N-Acetyl Arginine or N-Acetyl Glutamic Acid, each as described below.

N-Acetyl Methionine (C₇H₁₃NO₃S) is the substituted amino acid that conforms to the formula:

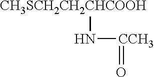

N-Acetyl Cysteine (C₅H₉NO₃S) is the substituted amino acid that conforms to the formula:

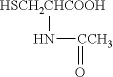

N-Acetyl Aspartic acid (C₆H₉NO₅) is the substituted amino acid that conforms to the formula:

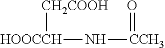

N-Acetyl Ornithine (C₇H₁₄N₂O₃) is the substituted amino acid that conforms to the formula:

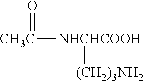

N-Acetyl Lysine (C₈H₁₆N₂O₃) is the substituted amino acid that conforms to the formula:

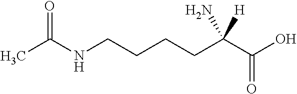

N-Acetyl Arginine (C₈H₁₆N₄O₃) is the substituted amino acid that conforms to the formula:

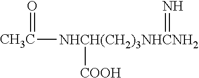

N-Acetyl Glutamic Acid (C₇H₁₁NO₅) is the substituted amino acid that conforms to the formula:

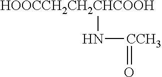

The at least one amino acid (or analogue thereof) is preferably completely reacted with an excess of glyoxylic acid, typically at a temperature of from about 140° F. to about 356° F.

In embodiments of the invention in which the hair-straightening complex includes two or more glyoxyloyl derivatives formed by reacting glyoxylic acid with two or more amino acids, and one or more of the amino acid(s) is/are sulfur-containing, the sulfur-containing amino acid(s) is/are reacted with glyoxylic acid in a first mixing vessel and the remaining amino acids are reacted with glyoxylic acid in a second mixing vessel. The reaction products formed in the first and second mixing vessels are then combined with the remaining components of the inventive hair-straightening complex, which, in turn, is combined with additional ingredients suitable for application to human hair to form the hair-straightening product of the present invention.

In embodiments of the invention in which the hair-straightening complex includes two or more glyoxyloyl derivatives formed by reacting glyoxylic acid with two or more amino acids, and one or more of the amino acid(s) has/have an n-acetyl group, the n-acetyl amino acid(s) is/are reacted with glyoxylic acid in a first mixing vessel and the remaining amino acids are reacted with glyoxylic acid in a second mixing vessel. The reaction products in the first and second mixing vessels are then combined with the remaining components of the hair-straightening complex of the present invention. The reaction products formed in the first and second mixing vessels are then combined with the remaining components of the inventive hair-straightening complex; this complex is then combined with additional ingredients suitable for application to human hair to form the hair-straightening product of the present invention.

In embodiments of the invention in which the hair-straightening complex includes two or more glyoxyloyl derivatives formed by reacting glyoxylic acid with two or more amino acids, and one or more of the amino acids has/have an n-acetyl group, and one or more of the amino acid(s) is/are sulfur-containing, the n-acetyl amino acid(s) is/are reacted with glyoxylic acid in a first mixing vessel, the sulfur-containing amino acid(s) is/are reacted in a second mixing vessel, and the remaining amino acids are reacted with glyoxylic acid in a third mixing vessel. The reaction products in the first, second and third mixing vessels are then combined with the remaining components of the hair-straightening complex; the complex is then combined with additional ingredients suitable for application to human hair to form the hair-straightening product of the present invention.

In one aspect of this embodiment, one of the amino acids that is reacted separately with glyoxylic acid is a sulfur-containing amino acid selected from the group consisting of Methionine, N-Acetyl Methionine, Cysteine, and N-Acetyl Cysteine.

In another aspect of this embodiment, one of the amino acids that is reacted separately with glyoxylic acid is an n-acetyl amino acid selected from the group consisting of N-Acetyl Methionine, N-Acetyl Cysteine, N-Acetyl Aspartic Acid, N-Acetyl Ornithine, N-Acetyl Lysine, N-Acetyl Arginine or N-Acetyl Glutamic Acid.

In one preferred embodiment, the hair-straightening complex is comprised of two or more glyoxyloyl derivatives formed by reacting glyoxylic acid with two or more amino acids, at least one of which is an essential amino acid. Included within this embodiment are hair-straightening complexes that are comprised of glyoxyloyl derivatives formed by reacting glyoxylic acid with: (i) at least one essential amino acid and at least one non-essential amino acid; (ii) at least one essential amino acid and at least conditional amino acid; (iii) at least one essential amino acid and at least one n-acetyl amino acid; (iv) at least one essential amino acid, at least one non-essential amino acid and either or both of a conditional amino acid and/or an n-acetyl amino acid; (v) at least one essential amino acid, at least one conditional amino acid and either or both of a non-essential amino acid and/or an n-acetyl amino acid; (vi) at least one essential amino acid, at least n-acetyl amino acid and either or both of a non-essential amino acid and/or a conditional amino acid.

In another preferred embodiment, the hair-straightening complex is comprised of two or more glyoxyloyl derivatives formed by reacting glyoxylic acid with two or more amino acids, at least one of which is a non-essential amino acid. Included within this embodiment are hair-straightening complexes that are comprised of glyoxyloyl derivatives formed by reacting glyoxylic acid with: (i) at least non-one essential amino acid and at least one essential amino acid; (ii) at least one non-essential amino acid and at least conditional amino acid; (iii) at least one non-essential amino acid and at least one n-acetyl amino acid; (iv) at least one non-essential amino acid, at least one essential amino acid and either or both of a conditional amino acid and/or an n-acetyl amino acid; (v) at least one non-essential amino acid, at least one conditional amino acid and either or both of an essential amino acid and/or an n-acetyl amino acid; and (vi) at least one non-essential amino acid, at least n-acetyl amino acid and either or both of an essential amino acid and/or a conditional amino acid.

In yet another preferred embodiment, the hair-straightening complex is comprised of two or more glyoxyloyl derivatives formed by reacting glyoxylic acid with two or more amino acids, at least one of which is a conditional amino acid. Included within this embodiment are hair-straightening complexes that are comprised of glyoxyloyl derivatives formed by reacting glyoxylic acid with: (i) at least one conditional amino acid and at least one essential amino acid; (ii) at least one conditional amino acid and at least non-essential amino acid; (iii) at least one conditional amino acid and at least one n-acetyl amino acid; (iv) at least one conditional amino acid, at least one essential amino acid and either or both of a non-essential amino acid and/or an n-acetyl amino acid; (v) at least one conditional amino acid, at least one non-essential amino acid and either or both of an essential amino acid and/or an n-acetyl amino acid; and (vi) at least one conditional amino acid, at least n-acetyl amino acid and either or both of an essential amino acid and/or a non-essential amino acid.

In a further preferred embodiment, the hair-straightening complex is comprised of two or more glyoxyloyl derivatives formed by reacting glyoxylic acid with two or more amino acids, at least one of which is an n-acetyl amino acid. Included within this embodiment are hair-straightening complexes that are comprised of glyoxyloyl derivatives formed by reacting glyoxylic acid with: (i) at least one n-acetyl amino acid and at least one essential amino acid; (ii) at least one n-acetyl amino acid and at least non-essential amino acid; and (iii) at least n-acetyl amino acid and at least one conditional amino acid; (iv) at least one n-acetyl amino acid, at least one essential amino acid and either or both of a non-essential amino acid and/or a conditional amino acid; (v) at least one n-acetyl amino acid, at least one non-essential amino acid and either or both of an essential amino acid and/or a conditional amino acid; and (vi) at least one n-acetyl amino acid, at least conditional amino acid and either or both of an essential amino acid and/or a non-essential amino acid.

In another embodiment, the hair-straightening complex is comprised of glyoxyloyl derivatives formed by reacting glyoxylic acid with at least one of each of an essential amino acid, a non-essential amino acid, a conditional amino acid, and a n-acetyl amino acid.

The glyoxyloyl derivative(s) is/are present at a concentration of from about 0.5% to about 50.0% wt/wt of the hair-straightening complex.

In certain preferred embodiments, where straightening of coarse, wavy or curly hair is desired for a period of “longer” period of time (at least one month), the glyoxyloyl derivative(s) is/are present at a “higher” glyoxyloyl derivative content (“GDC”)—from about 1.0% to about 30.0% wt/wt of the hair-straightening complex.

In other preferred embodiments, where straightening of coarse, wavy or curly hair is desired for a “shorter” period of time—days but not weeks—the glyoxyloyl derivative(s) is/are present at a “lower” GDC—from about 0.5% to about 20.0% wt/wt of the hair-straightening complex.

In still other preferred embodiments, where maintenance of straightening achieved by application of a “higher” GDC is desired, glyoxyloyl derivative(s) comprise from about 0.5% to about 5.0% wt/wt of the hair-straightening complex.

When the hair-straightening complex of the invention is included in a finished haircare product, the glyoxyloyl derivative(s) is/are present at a concentration of from about 0.025% to about 15.0% wt/wt, based on the total weight of the finished haircare product.

As used in the present application, the term “finished haircare product” is to be understood to mean a hair-straightening product of the present invention that consists essentially of inventive hair-straightening complex.

The hair-straightening complex of the invention includes as a required component at least one organic acid, preferably glyoxylic acid.

In embodiments where glyoxylic acid is the single organic acid that is part of the hair-straightening complex, glyoxylic acid is present at a concentration of from about 1.0% to about 60.0% wt/wt, based on the total weight of the hair-straightening complex; preferably from about 5.0% to about 55.0% wt/wt, and more preferably from about 20.0% to about 50.0% wt/wt.

In finished haircare products within the scope of this embodiment of the invention, glyoxylic acid is present at a concentration of from about 0.01% to about 18.0% wt/wt of the finished haircare product, and in more preferred embodiments from about 0.1% to about 10.0% wt/wt of the finished haircare product.

In other embodiments, the hair-straightening complex of the invention includes glyoxylic acid and a second organic acid, preferably a hydroxy acid or a keto acid, having six or fewer carbon atoms.

In embodiments containing a second organic acid (in addition to glyoxylic acid), the second organic acid is preferably selected from the group consisting of: acetic acid, acetolactic acid, citric acid, glutaric acid, glyceric acid, glycolic acid, hydroxy pyruvic acid, lactic acid, levulinic acid, oxopropanedioic acid, 2-oxopentanedioic acid, 3-oxopentanedioic acid, 2-oxobutanoic acid, malonic acid, mandelic acid and pyruvic acid.

In even more preferred embodiments containing a second organic acid (in addition to glyoxylic acid), the second organic acid is selected from the group consisting of acetolactic acid, glyceric acid, hydroxy pyruvic acid, oxopropanedioic acid, 2-oxopentanedioic acid, 3-oxopentanedioic acid, and pyruvic acid.

In embodiments containing a second organic acid, the second organic acid is present at a concentration of from about 0.025% to about 25.0% wt/wt, based on the total weight of the hair-straightening complex. In more preferred embodiments, the second organic acid is present at a concentration of from about 0.5 to about 25.0% wt/wt, based on the total weight of the hair-straightening complex, and in even more preferred embodiments from about 1.0% to about 10.0% wt/wt, based on the total weight of the hair-straightening complex.

An optional component of the hair-straightening complex of the present invention is at least one humectant, preferably a blend of more than one humectant.

In preferred embodiments, the at least one humectant is selected from the group consisting of Pyrrolidone Carboxylic Acid (PCA) and its salts, trimethylglycine, lactic acid, sodium lactate, glycerin, polyols, and unreacted, preferably non-keratin, amino acids. A particularly preferred humectant is the sodium salt of PCA.

The at least one (optional) humectant is preferably present at a concentration of from about 0.01% to about 5.0% wt/wt, based on the total weight of the finished haircare product; preferably from about 0.1% to about 2.5% wt/wt.

Urea and/or hydroxyethyl urea are humectants that may be incorporated in the hair-straightening complex of the present invention, but at levels that do not generate formaldehyde above the U.S. Occupational and Safety Administration “action level” (8-hour time weighted average of 0.5 ppm).

The hair-straightening complex of the present invention includes as fourth a required component from about 0.1% to 10.0% of one or more penetration enhancers, non-limiting examples of which include monoalkyl or dialkyl ether of sorbitol, laurocapram, oleic acid, dimethyl isosorbide and diethyl isosorbide.

In certain embodiments, the finished haircare product is a two-part emulsion—oil-in-water, water-in-oil, water-in-silicone, or silicone-in-water—that contains at least one surfactant, and preferably at least two surfactants, where the at least one surfactant is present in the finished haircare product at a concentration of from about 0.01% to about 20%, preferably from about 0.1% to about 15%, more preferably from about 0.5% to about 10%, by weight of the total finished haircare compositions. Surfactants may be cationic, anionic, zwitterionic, amphoteric, and non-ionic. If the composition is in the form of a shampoo or conditioner it will preferably contain anionic or cationic surfactants, respectively.

Suitable nonionic surfactants or emulsifiers include alkoxylated alcohols, or ethers, formed by the reaction of an alcohol with an alkylene oxide, usually ethylene or propylene oxide. Preferably the alcohol is a fatty alcohol having 6 to 30 carbon atoms. Examples of such ingredients include Beheneth 5-30, which is formed by the reaction of behenyl alcohol and ethylene oxide where the number of repeated ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on. Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol. Preferred are alkoxylated alcohols which are formed by the reaction of stearic acid, methyl glucose, and ethoxylated alcohol, otherwise known as PEG-20 methyl glucose sesquiisostearate.

Also suitable as the nonionic surfactant are alkyoxylated carboxylic acids, which are formed by the reaction of a carboxylic acid with an alkylene oxide or with a polymeric ether. The resulting products are described in paragraph [0172] of U.S. Pre-Grant Patent Application Publication 2004/0180020A1, the disclosure of which is incorporated herein by reference.

Other suitable nonionic surfactants include alkoxylated sorbitan and alkoxylated sorbitan derivatives. For example, alkoxylation, in particular, ethoxylation, of sorbitan provides polyalkoxylated sorbitan derivatives. Esterification of polyalkoxylated sorbitan provides sorbitan esters such as the polysorbates. Non-limiting xamples of such ingredients include Polysorbates 20-85, sorbitan oleate, sorbitan palmitate, sorbitan sesquiisostearate and sorbitan stearate.

Silicone surfactants—organosiloxane polymers having at least one hydrophilic radical and at least one lipophilic radical—are also suitable as nonionic surfactants. Non-limiting examples of silicone surfactants are commercially available from Dow Corning under the tradenames Dow Corning 3225C Formulation Aid, Dow Corning 190 Surfactant, Dow Corning 193 Surfactant, Dow Corning Q2-5200. Silicone surfactants of this type are generally referred to in the art as dimethicone copolyols or alkyl dimethicone copolyols.

Suitable cationic, anionic, zwitterionic, and amphoteric surfactants are disclosed in U.S. Pat. No. 5,534,265, the disclosure of which is incorporated, in pertinent part, by reference.

The finished haircare product may optionally, and preferably does, contain one or more conditioning agent(s) that adhere(s) to and improve(s) the feel and manageability of the hair. The conditioning agent(s) may be present at a concentration of from about 0.01% to about 30% wt/wt, more preferably from about 0.05% to about 20% wt/wt, and even more preferably from about 0.1% to 10% wt/wt, based on the total weight of the haircare product. Non-limiting examples of conditioning agents include polyquarternium polymers (polyquats), which will be understood by the person of ordinary skill in the art to vary by molecular structure and charge density (amount of positive charge/molecule). Other non-limiting examples of conditioning agents suitable for use in hair-straightening compositions of the present invention include silicones, higher alcohols (typically having 8 carbon atoms or more), hydrocarbon oils, natural triglycerides, polyolefins, fatty acid esters, fatty acid amides, and polyalkylene glycols.

The hair-straightening formulations of the present invention may optionally, and preferably do, contain one or more shine enhancers. Linear and cyclic dimethicones and esters having high refractive indexes (greater than 1.4) may be used as shine enhancers. A non-limiting example of an ester having a high refractive index suitable for use in the compositions of the present invention is propylene glycol dibenzoate.

Some shine enhancers also contribute to “slip” or “glide”. That is, they contribute to reducing tangling and pulling when hair is groomed after a hair-straightening treatment using the compositions of the present invention. Low molecular weight cyclic silicone compounds (e.g., pegylated dimethicones) and (poly)urethanes can be used as slip or glide enhancers.

Finished haircare products of the present invention typically also include organoleptic enhancers (colorants, fragrance) and preservatives.

Finished haircare products of the present invention consist essentially of Applicants' inventive hair-straightening complex as described above and have a pH of less than about 8.0, preferably a pH from about 0.5 to about 5.0, and still more preferably a pH of from about 1.0 to about 2.5.

Applicants' inventive method straightens human hair by treating the hair with a hair-straightening composition of the present invention as described above, followed by applying force to the so-treated hair, generally perpendicular to the length of the hair, using a flat surface, for example a flat iron, having a surface temperature of from about 380° F. to about 450° F.

An important inventive feature of the hair-straightening complex of the present invention, and finished haircare formulas containing the same, is that, when used in a hair-straightening procedure that involves application of heat (for example with a flat iron) no formaldehyde above the U.S. Occupational and Safety Administration “action level” (8-hour time weighted average of 0.5 ppm) is detected.

As noted above in the background of the invention, OSHA, both at federal and state levels, have warned that formaldehyde at levels having the potential for adverse health effects are released into the indoor environment when certain hair-straightening compositions are used in a hair-straightening procedure. Applicants have surprisingly found that, when the hair-straightening compositions of the present invention are used by a consumer or a stylist practicing the hair-straightening methods of the present invention, the user is exposed to formaldehyde at not more than 0.5 parts-per-million (ppm) formaldehyde, on an 8-hour time weighted basis (i.e., 8 hr. TWA<0.5 ppm).

Formaldehyde exposure (e.g., to a stylist, salon customer, or consumer using the inventive composition of the present invention at home) can be measured using either a passive vapor monitor (e.g., badge) and a forced-flow sorption tube (e.g., Draeger tube) techniques. Badges and tubes can be analyzed by National Institute of Occupational Safety Health method 2106 (Mar. 15, 2003). Exposure can measured over a period of about 2 hours and the results linearly extrapolated to arrive at an 8-hour TWA value.

A formaldehyde vapor monitor badge can be placed in the breathing zone of a person practicing the method of the present invention—for example on the collar of a shirt prior to beginning a hair-straightening procedure. After the procedure, the badge is sealed in a plastic bag and submitted to an AIHA accredited laboratory for analysis according to the NIOSH 2106 standard

The absence of formaldehyde can also be monitored using a sorption tube sampler (e.g., Draeger tube). The sorption tube is placed in the breathing zone of a person practicing the method of the present invention (e.g., on the collar of a shirt prior to a hair-straightening procedure). A constant pumping rate is used. Following the procedure, the tube is sealed in a plastic bag and submitted to an AIHA accredited laboratory for analysis in accordance with the NIOSH 2106 standard.

The hair-straightening complex of the present invention can be used both to achieve longer-lasting hair-straightening, for at least one month, preferably for a period of two to three months, as well as hair-straightening for shorter time frames (e.g., several days).

In one set of embodiments of hair-straightening methods of the invention in which longer-lasting hair-straightening is desired (e.g., for a period of at least one month, preferably for a period of at least two to three months), the finished haircare composition (lotion, serum, gel) that is applied to coarse, curly or wavy hair contains a “higher” glyoxyloyl derivative content—from about 5% to about 30% by weight of the complex, preferably at a concentration of from about 15% to about 25% by weight of the complex.

In these embodiments, the finished haircare product is applied to hair, preferably hair that has been dried, for a period at least 15 to 30 minutes, and thereafter contacted with a flat hair iron or similar heated hair-styling implement.

Embodiments of the invention in which longer-lasting hair-straightening is desired are preferably practiced in a hair salon or similar environment by a hair stylist or other haircare professional.

In particularly preferred embodiments, where longer-lasting hair-straightening is desired (e.g., for a period of two to three months), straightening of the hair is maintained/prolonged by application of a finished haircare composition (lotion, serum, gel) containing lower glyoxyloyl derivative content—glyoxyloyl derivative(s) comprise from about 0.5% to about 5% by weight of the complex, preferably from about 1.0% to about 3.0% by weight of the complex. This “maintenance” step is accomplished by application of the above-described a haircare product, preferably to dry hair, for a period at least one minute, preferably from 1 to 5 minutes, followed by application of a force generally perpendicular to the length of the hair using a flat surface heated at a temperature of from about 380° F. to about 450° F.

In a second set of embodiments, where straightening of coarse, wavy or curly hair is desired for a shorter period of time (e.g., for a period of at least 24 hours), the hair-straightening complex of the present invention is applied in a finished haircare composition (lotion, serum, gel) at a “lower” glyoxyloyl derivative content—from about 0.5% to about 5% by weight of the complex, preferably at a concentration of from about 1.0% to about 3.0% by weight of the complex.

The following formulation and applications examples are illustrative of the inventive hair-straightening complexes and formulations of the present invention. The components and specific ingredients of the example formulations are presented as being typical, and various modifications can be derived in view of the foregoing disclosure within the scope of the invention.

### Example 1

### Hair-Straightening Complex

| Ingredient | % (wt/wt) |
|---|---|
| Glyoxyloyl Derivative formed by mixing glyoxylic | 20.00 |
| acid with a mixture of L-Arginine, L-Aspartic | |
| Acid, Glycine, L-Alanine, L-Serine, L-Valine, L- | |
| Proline, L-Threonine, L-Isoleucine, and L- | |
| Histidine | |
| Glyoxylic Acid | 50.00 |
| Lactic Acid | 15.00 |
| Sodium PCA | 0.10 |
| Dimethyl Isosorbide | 5.00 |
| Water | Q.S. |
| Total: | 100.00 |

### Example 2

### Hair-Straightening Complex

| Ingredient | % (wt/wt) |
|---|---|
| Glyoxyloyl Derivative formed by (a) reacting | 20.00 |
| glyoxylic acid with a mixture of N-Acetyl Cysteine | |
| and Cysteine in a first mixing vessel, (b) reacting | |
| glyoxylic acid with Lysine in a second mixing | |
| vessel, and (c) combining the glyoxyloyl | |
| derivatives from the first and second vessels | |
| Glyoxylic Acid | 45.00 |
| Sodium PCA | 0.20 |
| Dimethyl Isosorbide | 5.00 |
| Water | Q.S. |
| Total : | 100.00 |

### Example 3

### Hair-Straightening Complex

| Ingredient | % (wt/wt) |
|---|---|
| Glyoxyloyl Derivative formed by reacting | 20.00 |
| glyoxylic acid with N-Acetyl Cysteine and | |
| Glyoxyolyl Cysteine | |
| Glyoxylic Acid | 40.00 |
| Sodium PCA | 0.20 |
| Dimethyl Isosorbide | 5.00 |
| Water | Q.S. |
| Total: | 100.00 |

### Example 4

### Hair Straightening Complex

| Ingredient | % (wt/wt) |
|---|---|
| Glyoxyloyl Derivative formed by (a) reacting | 10.00 |
| glyoxylic acid with N-Acetyl Cysteine in a first | |
| mixing vessel, (b) reacting glyoxylic acid with of | |
| L-Arginine, Glycine and L-Alanine in a second | |
| mixing vessel, and (c) combining the glyoxyloyl | |
| derivatives from the first and second vessels | |
| Glyoxylic Acid | 50.00 |
| Lactic Acid | 5.00 |
| Sodium PCA | 0.20 |
| Dimethyl Isosorbide | 5.00 |
| Water | Q.S. |
| Total: | 100.00 |

### Example 5

### Hair Straightening Complex

| Ingredient | % (wt/wt) |
|---|---|
| Glyoxyloyl Derivative formed by (a) reacting | 8.00 |
| glyoxylic acid with a mixture of N-Acetyl Cysteine | |
| and Cysteine in a first mixing vessel, (b) reacting | |
| glyoxylic acid with Cystine, and (c) combining the | |
| glyoxyloyl derivatives from the first and second | |
| vessels | |
| Glyoxylic Acid | 45.00 |
| Dimethyl Isosorbide | 5.00 |
| Sodium Lactate (optional) | 0.10 |
| Water | Q.S. |
| Total: | 100.00 |

### Example 6

### Hair-Straightening Complex

| Ingredient | % (wt/wt) |
|---|---|
| Glyoxyloyl Derivative formed by (a) reacting | 15.00 |
| glyoxylic acid with N-Acetyl Cysteine and | |
| Cysteine in a first mixing vessel, (b) reacting | |
| glyoxylic acid with Cystine and L-Arginine in a | |
| second mixing vessel, and (c) combining the | |
| glyoxyloyl derivatives from the first and second | |
| vessels | |
| Glyoxylic Acid | 50.0 |
| Lactic Acid | 2.00 |
| Urea | 0.20 |
| Dimethyl Isosorbide | 5.00 |
| Water | Q.S. |
| Total: | 100.00 |

### Example 7

### Hair-Straightening Complex

| Ingredient | % (wt/wt) |
|---|---|
| Glyoxyloyl Derivative formed by reacting | 20.00 |
| glyoxylic acid with N-Acetyl Cysteine and | |
| Cysteine | |
| Glyoxylic Acid | 40.00 |
| Lactic Acid | 0.50 |
| Dimethyl Isosorbide | 5.00 |
| Sodium Lactate (optional) | 0.10 |
| Water | Q.S. |
| Total: | 100.00 |

### Example 8

### Hair-Straightening Complex

| Ingredient | % (wt/wt) |
|---|---|
| Glyoxyloyl Derivative formed by mixing glyoxylic | 20.00 |
| acid with a mixture of L-Arginine, L-Aspartic | |
| Acid, Glycine, and B-Alanine | |
| Glyoxylic Acid | 40.50 |
| Lactic Acid | 2.00 |
| Sodium PCA | 0.10 |
| Dimethyl Isosorbide | 5.00 |
| Water | Q.S. |
| Total: | 100.00 |

### Example 9

### Hair-Straightening Complex

| Ingredient | % (wt/wt) |
|---|---|
| Glyoxyloyl Derivative formed by (a) reacting | 10.00 |
| glyoxylic acid with a mixture of N-Acetyl Cysteine | |
| and Cysteine in a first mixing vessel, (b) reacting | |
| glyoxylic acid with a mixture of L-Arginine, L- | |
| Aspartic Acid and Glycine in a second vessel, | |
| and (c) combining the glyoxyloyl derivatives from | |
| the first and second vessels | |
| Glyoxylic Acid | 43.50 |
| Lactic Acid | 2.00 |
| Dimethyl Isosorbide | 5.00 |
| Water | Q.S. |
| Total: | 100.00 |

### Example 10

### Hair-Straightening Complex

| Ingredient | % (wt/wt) |
|---|---|
| Glyoxyloyl Derivative formed by (a) reacting | 15.00 |
| glyoxylic acid with N-Acetyl Cysteine in a first | |
| mixing vessel, (b) reacting glyoxylic acid with a | |
| mixture of L-Arginine, L-Aspartic Acid, and B- | |
| Alanine in a second mixing vessel, and (c) | |
| combining the glyoxyloyl derivatives from the first | |
| and second | |
| Glyoxylic Acid | 50.00 |
| Lactic Acid | 5.00 |
| Sodium PCA | 0.20 |
| Dimethyl Isosorbide | 5.00 |
| Water | Q.S. |
| Total: | 100.00 |

### Example 11

### Hair-Straightening Shampoo

| Ingredient | % (wt/wt) |
|---|---|
| Cetrimonium Chloride | 15.00 |
| Cocamidopropyl Betaine | 10.00 |
| Laureth-5 | 5.00 |
| Cocamide MEA | 2.00 |
| Sodium Benzoate | 0.20 |
| Inventive Hair-Straightening Complex (from | 15.00 |
| any of Examples 1-10) | |
| Water | Q.S. |
| Total: | 100.00 |

### Example 12

### Hair-Straightening Emulsion

| Ingredient | % (wt/wt) |
|---|---|
| Cetearyl Alcohol | 2.00 |
| Shea Butter Cetyl Esters | 1.00 |
| Behentrimonium Methosulfate | 0.70 |
| Isopropyl Palmitate | 0.70 |
| Cethrimonium chloride 25% | 2.00 |
| Decamethylcyclopentasiloxane | 2.00 |
| Polyquaternium 67 | 0.20 |
| PEG-90 | 0.20 |
| MethylChloroIsothiazolinone/ | 0.10 |
| Methylisotiazolinone | |
| Inventive Hair-Straightening Complex | 20.00 |
| (from any of Examples 1-10) | |
| Water | Q.S. |
| Total: | 100.00 |

### Example 13

### Hair-Straightening Gel

| Ingredient | % |
|---|---|
| Sodium Acrylates Copolymer | 2.50 |
| Glycerin | 2.00 |
| Sunflower Oil | 4.00 |
| Phospholipids | 0.20 |
| Polygliceryl-10 Stearate | 0.20 |
| MethylChloroIsothiazolinone/ | 0.10 |
| Methylisotiazolinone | |
| Inventive Hair-Straightening Complex | 20.00 |
| (from any of Examples 1-10) | |
| Water | Q.S. |
| Total: | 100 |

### Example 14

### Hair-Straightening Solution

| Ingredient | % (wt/wt) |
|---|---|
| Glycerin | 5.00 |
| Cetrimonium Chloride (25%) | 1.00 |
| Polyquaternium 7 | 1.00 |
| MethylChloroIsothiazolinone/ | 0.10 |
| Methylisotiazolinone | |
| Inventive Hair-Straightening Complex | 15.00 |
| (from any of Examples 1-10) | |
| Water | Q.S. |
| Total: | 100.00 |

### Example 15

### Hair Straightening Serum (Maintenance or Short Term Straightening)

| Ingredient | % (wt/wt) |
|---|---|
| Cyclopentasiloxane (and) Dimethiconol | 65.00 |
| Cyclopentasiloxane | 29.00 |
| Lauryl PEG/PPG-18/18 Methicone | 2.00 |
| Hair Straightening Complex (from any of | 3.00 |
| Examples 1-10) | |
| Water | Q.S. |
| Total: | 100.00 |

### Example 16

### Hair Straightening Serum (Maintenance or Short Term Straightening)

| Ingredient | % (wt/wt) |
|---|---|
| Cyclopentasiloxane | 10.00 |
| Cyclopentasiloxane (and) Dimethiconol | 2.00 |
| Lecithin | 2.00 |
| Isopropyl Palmitate | 0.70 |
| Behentrimonium Methosulphate | 3.00 |
| Polyquaternium 55 | 5.0 |
| Panthenol | 0.20 |
| PEG-90 | 0.20 |
| MethylChloroIsothiazolinone/ | 0.10 |
| Methylisotiazolinone | |
| Hair Straightening Complex (from any of | 2.00 |
| Examples 1-10) | |
| Water | Q.S. |
| Total: | 100.00 |

Hair straightening serums containing a hair-straightening complex with a higher glyoxyloyl derivative content are preferably applied by a hair stylist or licensed hair professional in a salon or similar environment. Application of the hair-straightening complex in this manner can achieve hair-straightening for a period of months, typically 2-3, sometimes longer.

The hair-straightening serums of Examples 15 and 16 have a lower overall glyoxyloyl derivative content. Application of these “at-home” formulations can help maintain the straightening achieved by in-salon application of higher amounts of glyoxyloyl derivatives. In preferred embodiments, application of the “at home” hair-straightening serum, with a lower concentration of the hair-straightening complex, is applied at least once every 10 days.

Alternatively, the serums of Examples 15 and 16 containing a “lower” content of glyoxyloyl derivatives can also be applied by a hair stylist or licensed hair professional in a salon or similar environment to achieve a hair-straightening effects that lasts for a shorter duration (e.g., several days, as opposed to months).

### Example 17

### Comparative Haircare Blends

Nine haircare ingredients or blends of haircare ingredients, designated A-I in the table below, not including the hair-straightening complex of the present invention (as illustrated in Examples 1-3), were prepared for comparative testing (see Example 9 below):

| Ingredient | A | B | C | D | E | F | G | H | I |
|---|---|---|---|---|---|---|---|---|---|
| N-Acetyl Cysteine | | | | 1.00 | | | | 1.00 | 1.00 |
| L-Lysine HCl | | | | | | | 1.00 | 1.00 | |
| L-Cysteine | | | | | | 1.00 | | 1.00 | 1.00 |
| L-Arginine | | | | | | | | 1.00 | 1.000 |
| Lactic Acid | | | | | | | | | |
| Sodium PCA | | | 1.00 | | | | | | 1.00 |
| Glyoxylic Acid | 100.0 | | 99.00 | 99.00 | 95.00 | 94.00 | 94.00 | 91.00 | 91.00 |
| Dimethyl Isosorbide | | | | | 5.00 | 5.00 | 5.00 | 5.00 | 5.00 |
| Glyoxyloyl Carbocysteine + | | 100.0 | | | | | | | |
| Glyoxyloyl Keratin | | | | | | | | | |
| Aminoacids (Animal Origin)** | | | | | | | | | |

**Commercially available under the tradename Proliss from Aqia Quimica Industrial, Brazil, distributed in the United States by Cosphatech, LLC (Miami, Florida).

### Example 18

### Comparative Testing

Three hair-straightening complexes of the present invention, Examples 1-3 above, were added to a cationic emulsion base of Example 5, at 20%, forming three hair-straightening compositions. Nine comparative bases from Example 8, designated A-I, were added to the same cationic base, also at 20%, forming nine hair-straightening compositions. Each of the hair-straightening compositions was compared with respect to straightening and the number of washes before fading was observed. The results are presented in the following table:

| | Invention | | | Comparative Examples | | | | | | | | |
|---|---|---|---|---|---|---|---|---|---|---|---|---|
| | 1 | 2 | 3 | 9A | 9B | 9C | 9D | 9E | 9F | 9G | 9H | 9I |
| Straightening | ++ | ++ | ++ | + | ++ | + | Poor | ++ | ++ | Poor | Poor | ++ |
| # washes | 45 | >50 | >50 | 4 | 38 | 4 | NA | 11 | 12 | NA | NA | 7 |
| before fading | | | | | | | | | | | | |

### Example 19

### Accelerated Aging of the Hair-Straightening Composition

Samples of four hair-straightening compositions of the present invention, one sample from each of Examples 4-7 as described above, each sample of approximately 50 grams, are each placed into a clean glass jar. The jars are placed into a recirculating air oven maintained at 50° C. The jars are removed after four weeks, simulating approximately two-years storage under normal conditions. A sample of each of the formulations is analyzed for formaldehyde content according to NIOSH 2016M. The samples are found to contain <0.1 μg/g, which is considered below the quantification limit of the method used.

### Example 20

### Clinical Evaluation and Assessment of Hair and Hair Shaft After Treatment with the Hair-Straightening Composition of the Present Invention

Ten female test subjects, ages 18-50—five Causian, two African-American, and three Latin-American—having the following hair types are enrolled in a study to clinically evaluate and assess the efficacy of Example 7: one subject has coarse, wavy hair; one subject has fine, wavy hair; two subjects have coarse, curly hair; two subjects have fine, curly hair; two subjects have coarse, very curly hair; two subjects have fine, very curly hair. By “fine” hair is meant hair having a diameter from 40 to 75 microns. By “medium” hair is meant hair having a diameter from 75 to 100 microns. By “coarse” hair is meant hair having a diameter greater than 100 microns. “Wavy” hair is to be understood to mean hair exhibiting no to minimal curvature. “Curly” hair should be understood to mean hair fibers having obvious curvature with no overlap. By “very curly” hair is meant hair fibers having overlapping ringlets.

Prior to application of the hair-straightening composition, the hair of each subject is washed with a clarifying shampoo formulated to remove/strip oil, residue, and styling/condition agents from the hair. Each test subject's hair is dried with a blow dryer. The hair-straightening solution of Example 5 is placed in a bowl, and is applied to the dried hair with a brush, lock by lock, from root to tip. The hair is then covered with a plastic cap and the hair-straightening solution is allowed to remain on the hair for approximately twenty minutes, after which the hair is again dried with a blow drier. As a last step, the hair is straightened with five passes with a flat iron at a temperature of about 392° F. Each of the participants notice a significant decrease in curl and a significant increase in a shine, softness and manageability four weeks post-treatment.

### Example 21

### Passive Monitoring of Environmental Formaldehyde During Use of the Inventive Hair-Straightening Composition

A stylist is outfitted in the breathing area with a passive personal formaldehyde-monitoring device (badge). The stylist performs a hair-straightening procedure according to Application Example 1 using a hair-straightening composition of the present invention according to Example 7. The procedure is completed within approximately two hours. Following the procedure, the monitoring device is sealed in a plastic bag and is sent for analysis by NIOSH 2106. The 2-hour value is determined to be 0.01 ppm. An 8-hour TWA of 0.04 ppm is extrapolated from this value.

### Example 22

### Monitoring of Environmental Formaldehyde During Use of the Inventive Hair-Straightening Composition

A stylist performs a hair-straightening procedure according to Application Example 1 using a hair-straightening composition of the present invention according to Example 7. The stylist is outfitted in the breathing area with a formaldehyde sorption tube sampler. The tube is connected to a pump that draws air at a rate of 0.5 L/min. An identical tube, with pump, is positioned within the “treatment area”, approximately eight feet away from the stylist at a height of approximately three feet. The pumps are operated to sample the air during the hair-straightening procedure, which lasts about 2 hours. When the procedure is completed, the tubes are sealed in plastic bags and sent for analysis by NIOSH 2106.

Minor, non-statistically significant differences are measured between the formaldehyde values in the breathing area of the stylist and in the treatment area. A 2-hour average value is calculated and is used to extrapolate an 8 hour-TWA of formaldehyde during a treatment according to the method of the present invention, which is below the OSHA action level.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth hereinabove but rather that the claims be construed as encompassing all the features of patentable novelty which reside in the present invention, including all features which would be treated as equivalents thereof by those skilled in the art to which the invention pertains.

1. A hair-straightening complex consisting essentially of
(i) at least one glyoxyloyl derivative formed by reacting glyoxylic acid with at least one alpha, beta or gamma amino acid and/or an amino acid analogue, where the amino acid, or analogue thereof, is not derived from an animal source,
(ii) at least one organic acid
(iii) optionally, at least one humectant, and
(iv) at least one monoalkyl or dialkyl ether of sorbitol or 1,4-Dianhydrosorbitol.

2. The hair-straightening complex of claim 1 wherein the at least one organic acid is glyoxylic acid.

3. The hair-straightening complex of claim 2 containing at least two organic acids, wherein a first organic acid is glyoxylic acid, and a second organic acid has six or fewer carbon atoms.

4. The hair-straightening complex of claim 3 wherein the second organic acid is selected from the group of hydroxy acids and keto acids.

5. The hair-straightening complex of claim 1 wherein the at least one alpha, beta or gamma amino acid and/or an amino acid analogue is selected from the group consisting of essential amino acids, non-essential amino acids, conditional amino acids, n-acetyl amino acids, selenocysteine and beta-alanine.

6. A haircare composition containing a hair-straightening complex of claim 1.

7. The haircare composition of claim 6 further comprising at least one of
(i) a conditioning agent that adheres to and improves the feel and manageability of the hair or (ii) a shine enhancer.

8. A method of straightening hair comprising the steps of:
(i) providing a hair-straightening composition of claim 6,
(ii) applying the hair-straightening composition to the hair,
(iii) allowing the hair-straightening composition to reside on the hair
(iv) applying a force generally perpendicular to the length of the hair using a flat surface heated at a temperature of from about 380° F. to about 450° F.

9. The method of claim 8 wherein the hair is shampooed prior to step (i).

10. The method of claim 9 wherein a user or stylist who applies the hair-straightening composition and applies heat to hair treated with the hair-straightening composition is not exposed to formaldehyde at a level above 0.5 ppm on an 8-hour time-weighted average basis.

11. The method of claim 8 wherein the glyoxyloyl derivative(s) is/are present at a concentration of from about 1% to about 30% wt/wt of the hair straightening complex, the hair-straightening composition is applied to the hair for a period of time of from about 15 to 30 minutes and thereafter a force is applied to the hair in a manner that is generally perpendicular to the length of the hair using a flat surface heated at a temperature of from about 380° F. to about 450° F.

12. The method of claim 8 wherein the glyoxyloyl derivative(s) is/are present at a concentration of from about 0.5% to about 5% wt/wt of the hair straightening complex, the hair-straightening composition is applied to the hair for a period of time of from about 5 to 15 minutes and thereafter a force is applied to the hair in a manner that is generally perpendicular to the length of the hair using a flat surface heated at a temperature of from about 380° F. to about 450° F.
